(19) United States
(12) Patent Application Publication
(10) Pub. No.: US 2018/0132968 A1
(43) Pub. Date: May 17, 2018

(54) CONDITION SENSING AND AGENT RELEASING DEVICE TO INDUCE REMEDIATION OF THE DETECTED CONDITION OR MALADY

(71) Applicant: WINTER LION & COMPANY, INC., Winter Park, FL (US)

(72) Inventors: Eric Randolph Miller, Winter Park, FL (US); Manon Chin, Orlando, FL (US); Chris Donald Mason, Saint Cloud, FL (US)

(21) Appl. No.: 15/813,940

(22) Filed: Nov. 15, 2017

Related U.S. Application Data
(60) Provisional application No. 62/422,749, filed on Nov. 16, 2016.

(51) Int. Cl. A61C 1/00; A61C 19/06

(52) U.S. Cl. CPC A61C 1/0015; A61B 5/14539; A61C 2204/002; A61C 19/06

(57) ABSTRACT

An oral hygiene device may include a housing having first and second ends, the second end configured to be inserted into an opening of a user/animal, and a processor carried by the housing. The oral hygiene device may include a pH sensor carried by the housing at the second end, and a pH neutralization device carried by the housing and configured to emit neutralization agent into the opening of the user/animal. The processor may be coupled to the pH sensor and the pH neutralization device. The processor may be configured to detect a pH level within the opening of the user/animal, compare the pH level within the opening of the user/animal with a threshold pH level, and selectively control the pH neutralization device based upon the comparison.

### RELATED APPLICATION

This application is based upon prior filed copending provisional patent application No. 62/422,749 filed Nov. 16, 2016, the entire subject matter of which is incorporated herein by reference in its entirety.

### TECHNICAL FIELD

The present disclosure relates to the field of electronic devices, and, more particularly, to an oral hygiene device and related methods.

### SUMMARY

It is an object of the present disclosure to provide an oral hygiene device that efficiently provides for maintenance of oral hygiene.

This and other objects, features, and advantages in accordance with the present disclosure are provided by an oral hygiene device including a housing having first and second ends, the second end configured to be inserted into an opening of a user/animal, and a processor carried by the housing. The oral hygiene device includes a pH sensor carried by the housing at the second end, and a pH neutralization device carried by the housing and configured to emit neutralization agent into the opening of the user/animal. The processor is coupled to the pH sensor and the pH neutralization device. The processor is configured to detect a pH level within the opening of the user/animal, compare the pH level within the opening of the user/animal with a threshold pH level, and selectively control the pH neutralization device based upon the comparison. Advantageously, the oral hygiene device may provide robust pH level hygiene maintenance for the user/animal.

### BRIEF DESCRIPTION OF THE DRAWINGS

FIG. 1 is a schematic diagram of an oral hygiene device, according to the present disclosure.

FIG. 2 is a schematic top plan view of another embodiment of the oral hygiene device, according to the present disclosure.

FIG. 3 is a schematic side elevational view of the oral hygiene device of FIG. 2.

FIG. 4 is a flowchart of a method for operating the oral hygiene device of FIGS. 2-3.

### DETAILED DESCRIPTION

The present disclosure will now be described more fully hereinafter with reference to the accompanying drawings, in which several embodiments of the invention are shown. This present disclosure may, however, be embodied in many different forms and should not be construed as limited to the embodiments set forth herein. Rather, these embodiments are provided so that this disclosure will be thorough and complete, and will fully convey the scope of the present disclosure to those skilled in the art. Like numbers refer to like elements throughout.

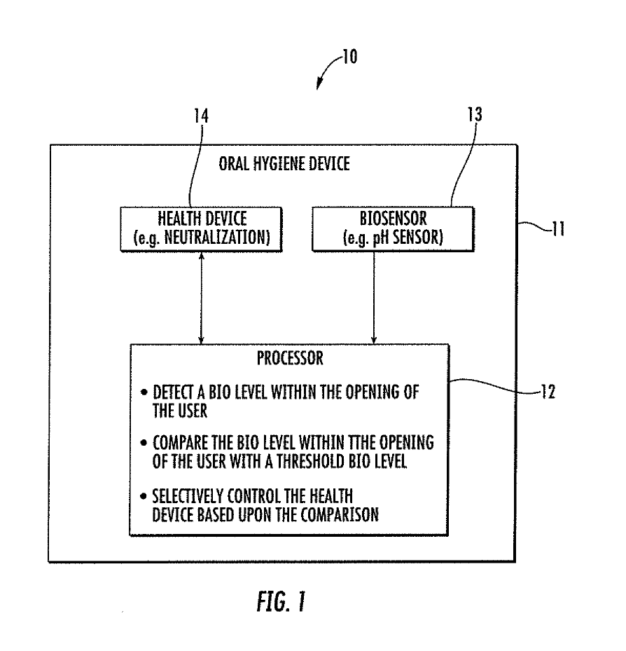

Referring initially to FIG. 1, an oral hygiene device 10 according to the present disclosure is now described. The oral hygiene device 10 illustratively includes a housing (e.g. an elongate housing) 11 having first and second ends. The second end is configured to be inserted into an opening of a user/animal, and the oral hygiene device 10 provides for maintenance of the pH levels (among other biological values) within the opening of the user/animal (i.e. controlling the acidity/basicity within the opening of the user/animal).

The oral hygiene device 10 illustratively includes a processor (e.g. system-on-chip processing integrated circuit) 12 carried by the housing 11. The oral hygiene device 10 illustratively includes a biosensor (e.g. a pH sensor) 13 carried by the housing 11 at the second end.

In some embodiments, the oral hygiene device 10 illustratively includes one or more additional biosensors. The additional biosensors may provide biological data related to pH levels, but may provide a very different data value, such as, for example, temperature, bacteria presence, deficiencies in normal values, inflammation, decay, infections, allergies and other health conditions. Also, the biosensors could detect analytes in saliva, breath, tissue, teeth, gums, micron and nano sized particles, which is not an exhaustive list and is provided only for exemplary purposes. In some embodiments, the biosensor 13 may comprise a multi-reading biosensor.

The oral hygiene device 10 illustratively includes a health device 14 carried by the housing 11. In some embodiments, the health device 14 may comprise a neutralization device and configured to emit a pH level neutralization agent into the opening of the user/animal. In certain embodiments, the health device 14 may comprise a health cartridge configured to emit agents, such as neutralization agents, good bacteria (probiotics) agents, inflammation reduction agents, hormone agents, vitamin agents, and other agents for health markers/needs or concerns that are detected by a sensor collected in the targeted orifice or dermatitis (skin). The agents will have varying flavor profiles. The agents may include pharmaceutical grade products, prescription or over the counter drugs, natural blends, vitamins, minerals, botanicals, liposomes, nano and micron formulations, energetics, veterinary formulas, hormones and other health targeted solutions. Of course, this list of agents is not exhaustive, and other agents could be used. Also, the health device 14 uses a variable or controlled release agent to optimize the timing of remediation process to most efficiently rectify the health condition.

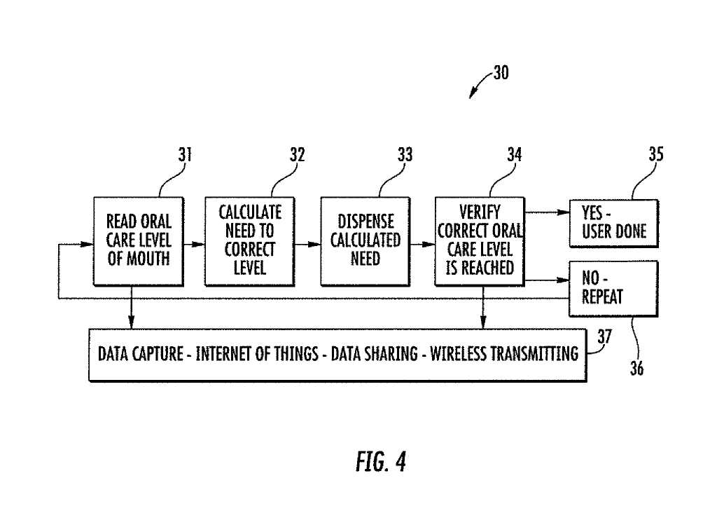

In some embodiments (e.g. FIGS. 3-4), the health device 14 is replaceable/interchangeable. For example, the health device 14 could be swapped out to provide varying sensing capabilities. In yet other embodiments, the health device 14 is static or fixed.

The processor 12 is coupled to the pH sensor 13 and the health device 14. The processor 12 is configured to detect a biological value (e.g. pH level) within the opening of the user/animal, compare the biological value within the opening of the user/animal with a threshold biological value, and selectively control the health device 14 based upon the comparison. In some embodiments, the biological value within the opening of the user/animal is compared to a desired biological value range.

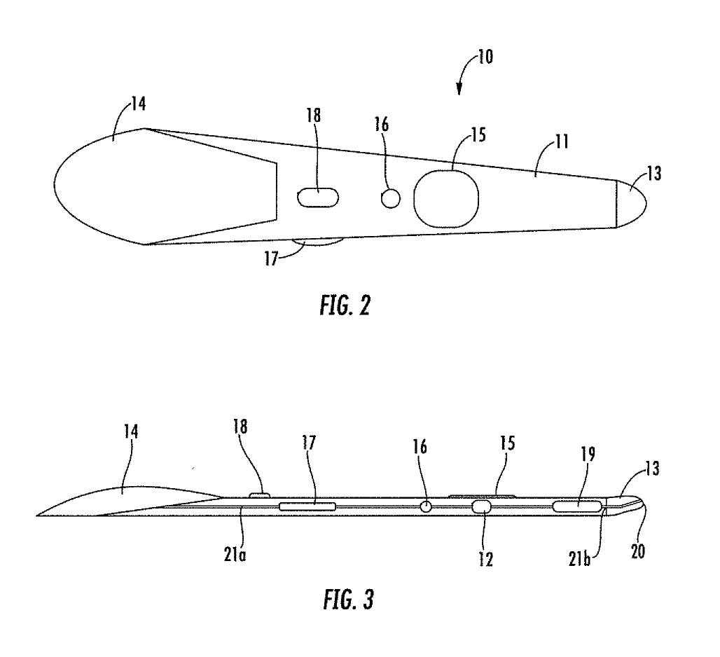

Referring now additionally to FIGS. 2-3, in the illustrated oral hygiene device 10, the health device 14 illustratively includes a pH neutralization device. The oral hygiene device 10 illustratively includes a pH range indicator 15 configured to generate an indication when the pH level within the opening of the user/animal is within a pH level range. In this embodiment, the pH range indicator 15 comprises a multi-color indicator (e.g. a multi-color light emitting diode (LED) module) to indicate whether the pH level within the opening of the user/animal is above, below, or within a desired pH level range. In this embodiment, the pH sensor 13 comprises a calibration free pH sensor. Also, this embodiment is intended for use in the oral opening of the user/animal, i.e. the mouth, but in other embodiments, the oral hygiene device 10 can be used in other bodily openings, such as the nasal opening, the vaginal opening, the ear opening, and the rectal opening, thereby providing a generic hygiene device. Also, it should be appreciated that the oral hygiene device 10 could be used on animals for similar functionality.

The oral hygiene device 10 illustratively includes a power supply 16 coupled to the processor 12, and an activation switch 17 coupled to the processor. The power supply 16 may comprise a replaceable battery power source, for example, and should provide several months or more of operation. Other embodiments may include an internal rechargeable battery source. The activation switch 17 is configured to receive input from the user, which causes the processor 12 to start the neutralization process. In this embodiment, the activation switch 17 comprises a soft-tip switch, for example, a rubberized toggle switch. The oral hygiene device 10 illustratively includes a neutralization process indicator (e.g. multi-color LED module) 18 configured to generate an indication when the neutralization process is in process and/or complete.

The oral hygiene device 10 illustratively includes an output nozzle (e.g. atomizer spray nozzle) 20 configured to emit the neutralization agent into the opening of the user/animal, and a pump device 19 coupled to the power supply 16 and configured to pressurize/direct the neutralization agent to the output nozzle. The oral hygiene device 10 illustratively includes first and second conduits 21a-21b coupled between the pH neutralization device 14 and the output nozzle 20 and providing fluid communication therebetween.

In certain embodiments, the pH neutralization device 14 comprises a replaceable pH neutralization cartridge, which is attached via a mechanical interlocking feature. In other embodiments, the pH neutralization device 14 may comprise a neutralization agent reservoir/tank/module, which may be refillable.

In some embodiments, the second end of the elongate housing 11 is replaceable for commercial application, i.e. high traffic use. The replaceable portion of the second end of the elongate housing 11 includes only the output nozzle 20 and a portion of the second conduit 21b, keeping per use costs down. The replaceable portion of the second end of the elongate housing 11 illustratively includes an interlocking feature, permitting a tight mechanical attachment to the first end of the elongate housing.

In some embodiments, the oral hygiene device 10 may comprise a wireless transceiver (e.g. IEEE 802.11 transceiver, a Bluetooth transceiver, or a near field communications (NFC) transceiver) (not shown) configured to connect to an external network (e.g. the Internet) for data transmission to web services. In some applications, the pH level data can be stored in the web services.

In other embodiments, the oral hygiene device 10 may comprise a wired data interface (not shown) coupled to the processor 12, and in rechargeable battery embodiments, the power supply 16. The wired data interface may comprise a universal serial bus (USB) interface, such as micro USB, and the wired data interface is configured to permit a wired connection to the processor 12. Via either the wired data interface or the wireless transceiver, the processor 12 may receive software updates to the neutralization algorithm.

The pH range indicator 15 and the neutralization process indicator 18 may also generate audio indications. In wireless embodiments, the indicators may send wireless alerts to external devices, such as generating a notification on an associate cellular device.

Referring now additionally to FIG. 4, a flowchart 30 illustrates a method for operating the oral hygiene device 10. The method includes inserting the second end of the elongate housing 11 into the opening of the user/animal (Blocks 31, 37), and operating the pH sensor 13 and the processor 12 for detecting a pH level within the opening of the user/animal. The method includes operating the processor 12 to process the pH level within the opening of the user/animal with a neutralization algorithm to determine if a neutralization process is needed, and if so, determining an amount of neutralization agent to be dispensed (Block 32).

The method includes operating the processor 12 and the pump device 19 to emit the amount of the neutralization agent into the opening of the user/animal (Block 33). The method also includes operating the pH sensor 13 and the processor 12 for detecting a new pH level within the opening of the user/animal (Block 34), and operating the processor to process the new pH level within the opening of the user/animal with the neutralization algorithm to determine if an additional neutralization process is needed (Block 35). The processor 12 is configured to restart the neutralization process if the additional neutralization process is needed, and to otherwise, end the neutralization process (Block 36).

Many modifications and other embodiments of the present disclosure will come to the mind of one skilled in the art having the benefit of the teachings presented in the foregoing descriptions and the associated drawings. Therefore, it is understood that the present disclosure is not to be limited to the specific embodiments disclosed, and that modifications and embodiments are intended to be included within the scope of the appended claims.

That which is claimed is:

1. An oral hygiene device comprising:
a housing having first and second ends, said second end configured to be inserted into an opening of a user/animal;
a processor carried by said housing;
a pH sensor carried by said housing at the second end;
a pH neutralization device carried by said housing and configured to emit neutralization agent into the opening of the user/animal;
said processor coupled to said pH sensor and said pH neutralization device, said processor configured to
detect a pH level within the opening of the user/animal,
compare the pH level within the opening of the user/animal with a threshold pH level, and
selectively control the pH neutralization device based upon the comparison.

2. The oral hygiene device of claim 1 further comprising at least one additional biosensor carried by said housing.

3. The oral hygiene device of claim 1 further comprising a pH range indicator configured to indicate whether the pH level within the opening of the user/animal is within a desired pH level range.

4. The oral hygiene device of claim 1 further comprising a power supply coupled to said processor, and an activation switch coupled to said processor.

5. The oral hygiene device of claim 4 wherein said power supply comprises a battery power source.

6. The oral hygiene device of claim 1 further comprising an output nozzle configured to emit the neutralization agent into the opening of the user/animal.

7. The oral hygiene device of claim 6 further comprising first and second conduits coupled between said pH neutralization device and said output nozzle and providing fluid communication therebetween.

8. The oral hygiene device of claim 1 further comprising a wireless transceiver configured to connect to an external network for data transmission to web services.

9. The oral hygiene device of claim 1 further comprising a neutralization process indicator configured to generate an indication of progress in the neutralization process.
